(19) United States
(12) Patent Application Publication
(10) Pub. No.: US 2003/0125600 A1
(43) Pub. Date: Jul. 3, 2003

(54) Moving system and moving method therefor

(75) Inventor: Seido Nagano, Tokyo (JP)

Correspondence Address: SUGHRUE MION, PLLC, 2100 PENNSYLVANIA AVENUE, N.W., WASHINGTON, DC, 20037, US

(73) Assignee: NEC CORPORATION

(21) Appl. No.: 10/329,480

(22) Filed: Dec. 27, 2002

(30) Foreign Application Priority Data
Dec. 27, 2001 (JP) 395818/2001

(51) Int. Cl. A61N 1/00

(52) U.S. Cl. 600/15

(57) ABSTRACT

A moving system includes an oscillation portion and a conversion portion. The oscillation portion causes oscillation in accordance with a natural frequency by repeating expansion and contraction. The conversion portion converts the oscillation of the oscillation portion into rectilinear movement in one direction. A moving method is also disclosed.

### BACKGROUND OF THE INVENTION

The present invention relates to a moving system which converts the oscillation of an oscillator into a thrust for rectilinear movement and a moving method therefor.

Current moving systems normally use the rotational movement of a power component such as a motor as a thrust for movement.

However, size reduction of electronic devices is now rapidly progressing, and it is difficult for a device to incorporate a complex power component such as a motor. Simplification and size reduction of moving systems are problems to be solved.

### SUMMARY OF THE INVENTION

It is an object of the present invention to provide a simple and compact moving system and a moving method therefor.

In order to achieve the above object, according to the present invention, there is provided a moving system comprising oscillation means for causing oscillation in accordance with a natural frequency by repeating expansion and contraction, and conversion means for converting oscillation of the oscillation means into rectilinear movement in one direction.

### BRIEF DESCRIPTION OF THE DRAWINGS

FIGS. 1A to 1D are side views for explaining the arrangement and operation of a moving system according to the first embodiment of the present invention;

FIGS. 2A and 2B are plan and side views, respectively, of a moving system according to the second embodiment of the present invention;

FIGS. 3A to 3D are plan views for explaining the operation of the moving system shown in FIGS. 2A and 2B;

FIG. 4 is a side view of a moving system according to the third embodiment of the present invention;

FIGS. 5A to 5D are side views for explaining the operation of the moving system shown in FIG. 4;

FIGS. 6A and 6B are side views for explaining the arrangement and operation of a moving system according to the fourth embodiment of the present invention;

FIGS. 7A and 7B are plan and side views, respectively, showing the arrangement of a moving system according to the fifth embodiment of the present invention;

FIGS. 8A to 8C are plan views for explaining the operation of the moving system shown in FIGS. 7A and 7B; and

FIGS. 9A to 9D are plan views for explaining the arrangement and operation of a moving system according to the sixth embodiment of the present invention.

### DESCRIPTION OF THE PREFERRED EMBODIMENTS

The present invention will be described below in detail with reference to the accompanying drawings.

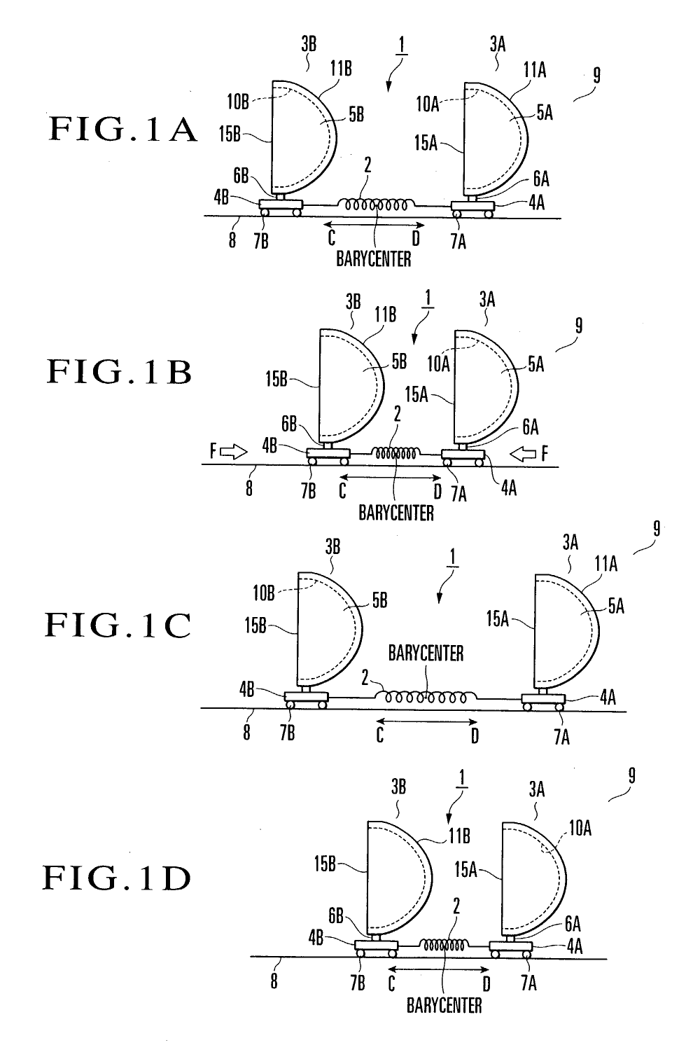

A moving system according to the first embodiment of the present invention will be described with reference to FIGS. 1A to 1D. FIG. 1A shows a moving system 1 which is set in an equilibrium state without expansion of a spring 2 and arranged on a solid surface 8. The moving system 1 according to this embodiment has the expandable spring 2 having a natural frequency and drag adjusting units 3A and 3B fixed to the two ends of the spring 2, as shown in FIG. 1A.

The drag adjusting units 3A and 3B comprise plate-shaped supports 4A and 4B horizontally placed, and hemispherical bodies 5A and 5B attached to the supports 4A and 4B through direction control plates 6A and 6B, respectively. Small casters 7A and 7B are attached to the lower surfaces of the supports 4A and 4B. The casters 7A and 7B reduce the friction between the moving system 1 and the solid surface 8 when the moving system 1 moves on the solid surface 8. The moving system 1 moves in a medium 9 made of a fluid such as a liquid or/and a gas, which fill the space on the solid surface 8. The barycenter of the moving system 1 is located almost at the center of the spring 2.

The hemispherical bodies 5A and 5B have cavities inside and openings 15A and 15B that are open to the medium 9. The hemispherical body 5A has inner and outer surfaces 10A and 11A. The hemispherical body 5B has inner and outer surfaces 10B and 11B. The direction control plates 6A and 6B can rotate on the supports 4A and 4B. When the direction control plates 6A and 6B rotate by 180°, the opening directions of the openings 15A and 15B of the hemispherical bodies 5A and 5B are reversed.

The operation of the moving system 1 when the openings 15A and 15B of the hemispherical bodies 5A and 5B are directed in a direction C (from the drag adjusting unit 3A to the drag adjusting unit 3B) will be described next.

In the moving system 1 set in the equilibrium state, when forces F are applied to the outer ends of the supports 4A and 4B such that the drag adjusting units 3A and 3B come close to each other, the spring 2 contracts, as shown in FIG. 1B. When the spring 2 contracts, the hemispherical body 5A receives, on its inner surface 10A, stress from the medium 9 through the opening 15A. Hence, the drag force from the medium 9 against the movement of the drag adjusting unit 3A in the direction C is large. On the other hand, the hemispherical body 5B only receives, on its outer surface 11B, stress from the medium 9 when the spring 2 contracts. Hence, the drag force from the medium 9 against the movement of the drag adjusting unit 3B in a direction D (from the drag adjusting unit 3B to the drag adjusting unit 3A) is small. For this reason, the moving distance of the drag adjusting unit 3A in the direction C is shorter than that of the drag adjusting unit 3B in the direction D. Accordingly, the barycenter of the moving system 1 moves in the direction D.

In this state, when the forces F at the outer ends of the supports 4A and 4B are canceled, the spring 2 expands, as shown in FIG. 1C. When the spring 2 expands, the hemispherical body 5A receives, on its outer surface 11A, stress only from the medium 9. Hence, the drag force from the medium 9 against the movement of the drag adjusting unit 3A in the direction D is small. On the other hand, the hemispherical body 5B receives, on its inner surface 10B, stress from the medium 9 through the opening 15B. Hence, the drag force from the medium 9 against the movement of the drag adjusting unit 3B in the direction C is large. For this reason, the moving distance of the drag adjusting unit 3A in the direction D when the forces F are canceled is longer than that of the drag adjusting unit 3B in the direction C. Accordingly, the barycenter of the moving system 1 moves in the direction D.

Next, the spring 2 contracts in accordance with its natural frequency, as shown in FIG. 1D. When the spring 2 contracts, the hemispherical body 5A receives, on its inner surface 10A, stress from the medium 9 through the opening 15A. Hence, the drag force from the medium 9 against the movement of the drag adjusting unit 3A in the direction C is large. On the other hand, the hemispherical body 5B only receives, on its outer surface 11B, stress from the medium 9. Hence, the drag force from the medium 9 against the movement of the drag adjusting unit 3B in the direction D is small. For this reason, the moving distance of the drag adjusting unit 3A in the direction D is shorter than that of the drag adjusting unit 3B in the direction C. Accordingly, the barycenter of the moving system 1 moves in the direction D.

Next, the spring 2 expands in accordance with its natural frequency. When the spring 2 expands, the drag adjusting unit 3A largely moves in the direction D while the drag adjusting unit 3B slightly moves in the direction C, as in FIG. 1C. Hence, the barycenter of the moving system 1 moves in the direction D.

Every time the spring 2 contracts/expands in accordance with its natural frequency, the barycenter of the moving system 1 always moves in the direction D. The entire moving system 1 moves in the direction in which the outer surfaces 11A and 11B of the drag adjusting units 3A and 3B are directed. The movement of the moving system 1 continues until the external force F is consumed as heat by the friction generated between the moving system 1 and the medium 9 by contraction/expansion of the spring 2.

When the direction control plates 6A and 6B are rotated by 180°, the openings 15A and 15B are directed in the direction D. This is equivalent to 180° rotation of the entire moving system 1. In this case, the moving system 1 moves in the direction C, as is apparent from the above description. According to the first embodiment, the drag adjusting units 3A and 3B convert the oscillation of the spring 2 into rectilinear movement.

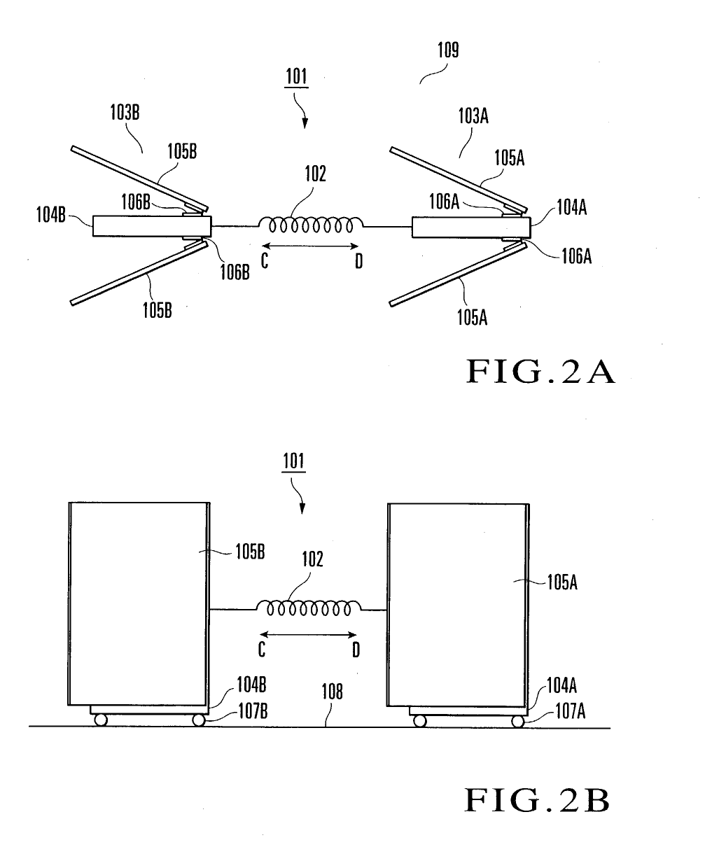

A moving system according to the second embodiment of the present invention will be described with reference to FIGS. 2A and 2B.

As shown in FIG. 2A, a moving system 101 according to this embodiment has a spring 102 and drag adjusting units 103A and 103B fixed to the two ends of the spring 102. The drag adjusting units 103A and 103B comprise thick plate-shaped supports 104A and 104B each of which is vertically placed such that the two surfaces become parallel to the moving direction, and pairs of plate-shaped bodies 105A and 105B attached to the D-direction ends of the both surfaces of the supports 104A and 104B through hinges 106A and 106B, respectively. The pairs of hinges 106A and 106B can open within the range of an angle α to 90°. The angle α can take any value as long as it is smaller than 90°, though the angle α is preferably about 2° to 30°. The plate-shaped bodies 105A and 105B are fixed to the hinges 106A and 106B. Hence, the angle made by the pair of plate-shaped bodies 105A or the pair of plate-shaped bodies 105B ranges from the minimum angle 2α to the maximum angle of 180°. In an equilibrium state without expansion of the spring, both the pair of plate-shaped bodies 105A and the pair of plate-shaped bodies 105B make the angle 2α or an arbitrary angle. The pairs of plate-shaped bodies 105A and 105B open in the same direction.

Small casters 107A and 107B are attached to the lower ends of the supports 104A and 104B, as shown in FIG. 2B. The casters 107A and 107B reduce the friction between the moving system 101 and a solid surface 108 when the moving system 101 moves on the solid surface 108. The moving system 101 moves in a medium 109 made of a fluid such as a liquid or/and a gas, which fill the space on the solid surface 108.

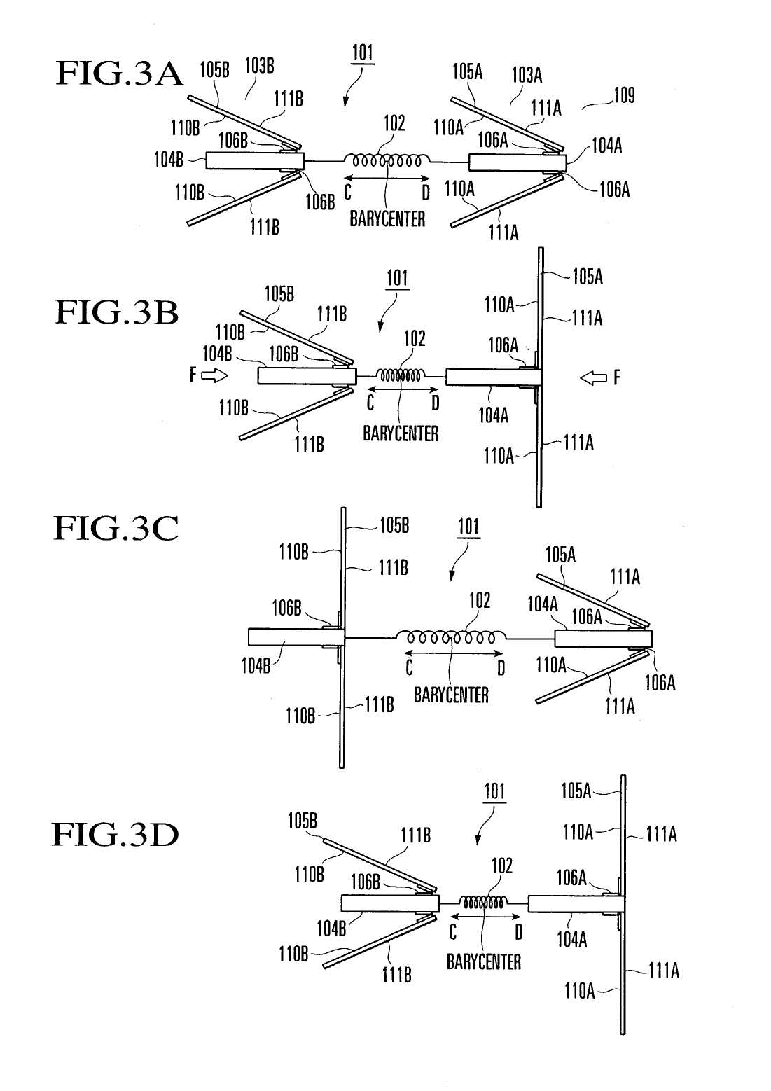

FIG. 3A shows the moving system 1 which is set in the equilibrium state without expansion of the spring 102 and arranged in the medium 109. Referring to FIG. 3A, each of the plate-shaped bodies 105A has inner and outer surfaces 110A and 111A. Each of the plate-shaped bodies 105B has inner and outer surfaces 110B and 111B. The plate-shaped bodies 105A and 105B make an angle close to the minimum angle 2α. The barycenter of the moving system 101 is located almost at the center of the spring 102.

When forces F are applied to the outer ends of the supports 104A and 104B of the moving system 101 in the equilibrium state such that the drag adjusting units 103A and 103B come close to each other, the spring 102 contracts, as shown in FIG. 3B. As the spring 102 contracts, the plate-shaped bodies 105A open to the maximum angle of 180° upon receiving, on their inner surfaces 110A, stress from the medium 109. On the other hand, the plate-shaped bodies 105B close to the minimum angle 2α upon receiving, on their outer surfaces 111B, stress from the medium 109 as the spring 102 contracts. In this case, the plate-shaped bodies 105A open up to the maximum angle of 180° and therefore receive a large force from the medium 109. The plate-shaped bodies 105B close to the minimum angle 2α and therefore receive a small force from the medium 109. Hence, the moving distance of the drag adjusting unit 103A in a direction C when the forces F are applied is shorter than that of the drag adjusting unit 103B in the direction D. For this reason, the barycenter of the moving system 101 moves in the direction D.

In this state, when the forces F are canceled, the spring 102 expands. As the spring 2 expands, the plate-shaped bodies 105A close to the minimum angle 2α upon receiving, on their outer surfaces 111A, stress from the medium 109. On the other hand, the plate-shaped bodies 105B open to the maximum angle of 180° upon receiving, on their inner surfaces 110B, stress from the medium 109 as the spring 102 expands. In this case, the plate-shaped bodies 105A close to the minimum angle 2α and therefore receive a small force from the medium 109. The plate-shaped bodies 105B open to the maximum angle of 180° and therefore receive a large force from the medium 109. Hence, the moving distance of the drag adjusting unit 103A in the direction D when the forces F are canceled is longer than that of the drag adjusting unit 103B in the direction C. For this reason, the barycenter of the moving system 1 moves in the direction D.

Next, the spring 102 contracts in accordance with its natural frequency, as shown in FIG. 3D. As the spring 102 contracts, the plate-shaped bodies 105A open to the maximum angle of 180° upon receiving, on their inner surfaces 110A, stress from the medium 109. On the other hand, the plate-shaped bodies 105B close to the minimum angle 2α upon receiving, on their outer surfaces 111B, stress from the medium 109 as the spring 102 contracts. In this case, the plate-shaped bodies 105A open up to the maximum angle of 180° and therefore receive a large force from the medium 109. The plate-shaped bodies 105B close to the minimum angle 2α and therefore receive a small force from the medium 109. Hence, the moving distance of the drag adjusting unit 103A in the direction C is shorter than that of the drag adjusting unit 103B in the direction D. For this reason, the barycenter of the moving system 101 moves in the direction D.

Next, the spring 102 expands in accordance with its natural frequency. When the spring 102 expands, the drag adjusting unit 103A largely moves in the direction D while the drag adjusting unit 103B slightly moves in the direction C, as in FIG. 3C. For this reason, the barycenter of the moving system 101 moves in the direction D.

The spring 102 repeatedly contracts/expands in accordance with its natural frequency. At this time, since the barycenter of the moving system 101 always moves in the direction D, the entire moving system 101 moves in the direction D. The movement of the moving system 101 continues until the external force F is consumed as heat by the friction generated between the moving system 101 and the medium by contraction/expansion of the spring 102.

In the above description, the opening angle of the hinges 106A and 106B ranges from α to 90° such that the plate-shaped bodies 105A and 105B open in only one of the moving directions. However, the plate-shaped bodies 105A and 105B may open in both of the moving directions. More specifically, when the opening angle of the hinges 106A and 106B is set in two steps, i.e., from α to 90° and from 90° to (180°-α), the moving system 101 on the solid surface 108 can also move in the direction C.

As described above, in the second embodiment as well, the drag adjusting units 103A and 103B convert the oscillation of the spring 102 into rectilinear movement, as in the first embodiment. In the second embodiment, additionally, each drag adjusting unit has a means for changing the drag force from the medium while the spring expands/contracts. For this reason, the moving distance ratio between the two drag adjusting units can be made higher than that in the first embodiment.

In the above-described first and second embodiments, the moving system moves on the solid surface. However, the present invention is not limited to this. For example, when the specific gravity of the entire moving system is designed to be equal to that of the medium, the moving system can move from an arbitrary point in the medium in an arbitrary direction.

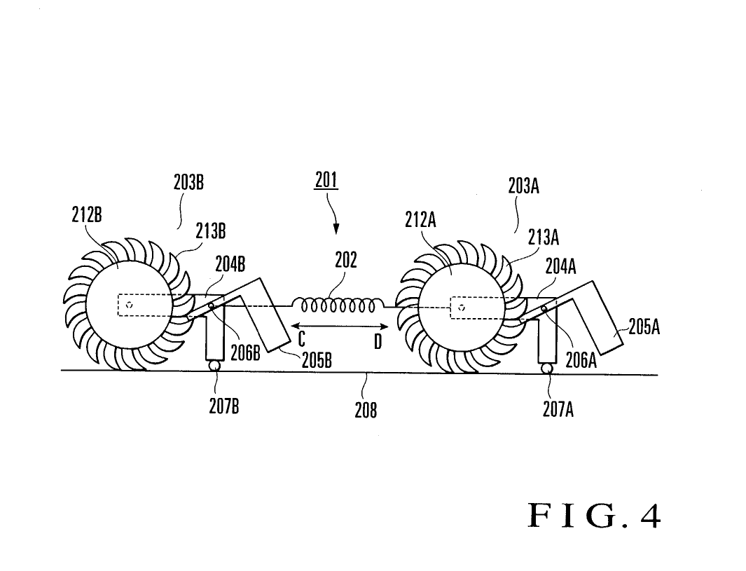

A moving system according to the third embodiment of the present invention will be described next with reference to FIG. 4.

A moving system 201 according to this embodiment has a spring 202 and friction adjusting units 203A and 203B fixed to the two ends of the spring 202, as shown in FIG. 4. The friction adjusting units 203A and 203B have supports 204A and 204B each having an L shape when viewed from a side, circular-saw-shaped wheels 212A and 212B rotatably supported by the supports 204A and 204B, and L-shaped plate-shaped bodies 205A and 205B pivotally supported by the supports 204A and 204B by pins 206A and 206B, respectively.

The supports 204A and 204B are constituted by horizontal portions which support the wheels 212A and 212B at the C-direction end portions, and vertical portions which have upper end portions connected to the D-direction end portions of the supports 204A and 204B and small casters 207A and 207B attached to the lower surfaces of the lower end portions. The plate-shaped bodies 205A and 205B are constituted by arm portions supported by the pins 206A and 206B and brake portions connected to the arm portions at an angle of 90°.

In an equilibrium state without expansion of the spring 202, the distal ends of the arm portions of the plate-shaped bodies 205A and 205B are inserted into serrate portions 213A and 213B of the wheels 212A and 212B. When the wheels 212A and 212B rotate clockwise, the distal end portions of the arm portions of the plate-shaped bodies 205A and 205B are pressed downward by the back surfaces of the serrate portions 213A and 213B, and the distal end portions of the brake portions of the plate-shaped bodies 205A and 205B are separated from a solid surface 208. On the other hand, when the wheels 212A and 212B rotate counterclockwise, the distal ends of serrate portions 213A and 213B press the distal ends of the arm portions of the plate-shaped bodies 205A and 205B upward. For this reason, the brake portions of the plate-shaped bodies 205A and 205B move downward to bring their distal ends into contact with the solid surface 208 to press the solid surface 208.

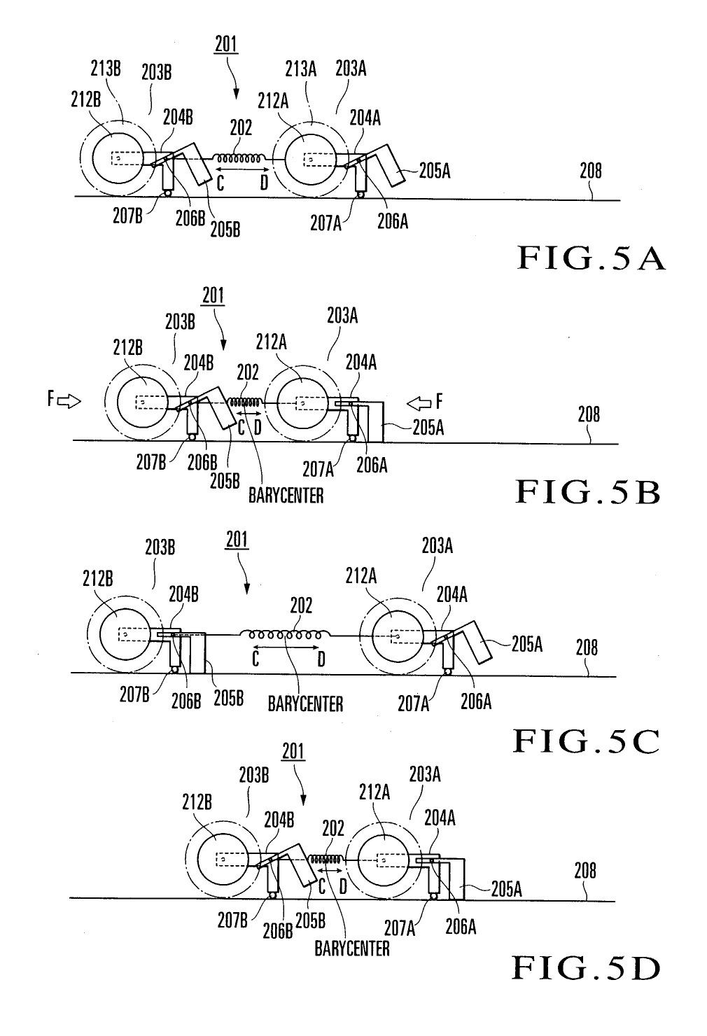

FIG. 5A shows the moving system 201 which is set in the equilibrium state without expansion of the spring 202 and arranged on the solid surface 208. The barycenter of the moving system 201 is located almost at the center of the spring 202. Referring to FIGS. 5A to 5D, the serrate portions 213A and 213B of the wheels 212A and 212B are represented by circumscribed circles of alternate long and short dashed lines.

Forces F are applied to the outer ends of the supports 204A and 204B of the moving system 201 in the equilibrium state such that the friction adjusting units 203A and 203B come close to each other. When the spring 202 contracts due to the applied forces F, the wheel 212A is going to rotate counterclockwise. However, as soon as the wheel 212A rotates, the brake portion of the plate-shaped body 205A is strongly pressed against the solid surface 208. On the other hand, the wheel 212B rotates clockwise. The brake portion of the plate-shaped body 205B is separated from the solid surface 208. When the brake portion of the plate-shaped body 205A is strongly pressed against the solid surface 208, a large frictional force acts between the solid surface 208 and the brake portion of the plate-shaped body 205A. On the other hand, no frictional force acts between the plate-shaped body 205B and the solid surface 208 because the brake portion of the plate-shaped body 205A is separated from the solid surface 208. Hence, the friction adjusting unit 203A slightly moves in the direction C while the friction adjusting unit 203B largely moves in the direction D. For this reason, the barycenter of the moving system 201 moves in the direction D.

In this state, when the forces F are canceled, the spring 202 expands. When the spring 202 expands, the wheel 212A rotates clockwise to separate the brake portion of the plate-shaped body 205A from the solid surface 208, as shown in FIG. 5C. On the other hand, when the spring 202 expands, the wheel 212B is going to rotate counterclockwise. However, as soon as the wheel 212B rotates, the brake portion of the plate-shaped body 205B is strongly pressed against the solid surface 208. On the other hand, since the brake portion of the plate-shaped body 205A is separated from the solid surface 208, no frictional force acts between the plate-shaped body 205A and the solid surface 208. Since the brake portion of the plate-shaped body 205B is strongly pressed against the solid surface 208, a strong frictional force acts between the plate-shaped body 205B and the solid surface 208. Hence, when the forces F are canceled, the friction adjusting unit 203A largely moves in the direction D while the friction adjusting unit 203B slightly moves in the direction C. For this reason, the barycenter of the moving system 201 moves in the direction D.

Next, the spring 202 contracts in accordance with its natural frequency, as shown in FIG. 5D. When the spring 202 contracts, the wheel 212A is going to rotate counterclockwise. However, as soon as the wheel 212A rotates, the brake portion of the plate-shaped body 205A is strongly pressed against the solid surface 208. On the other hand, when the spring 202 contracts, the wheel 212B rotates clockwise. The brake portion of the plate-shaped body 205B is separated from the solid surface 208. Since the brake portion of the plate-shaped body 205A is strongly pressed against the solid surface 208, a large frictional force acts between the plate-shaped body 205A and the solid surface 208. Since the brake portion of the plate-shaped body 205B is separated from the solid surface 208, no frictional force acts between the plate-shaped body 205B and the solid surface 208. Hence, the friction adjusting unit 203A slightly moves in the direction C while the friction adjusting unit 203B largely moves in the direction D. For this reason, the barycenter of the moving system 201 moves in the direction D.

Next, the spring 202 expands in accordance with its natural frequency. When the spring 202 expands, the friction adjusting unit 203A largely moves in the direction D while the friction adjusting unit 203B slightly moves in the direction C, as in FIG. 5C. For this reason, the barycenter of the moving system 201 moves in the direction D.

The spring 202 repeatedly contracts/expands in accordance with its natural frequency. At this time, since the barycenter of the moving system 201 always moves in the direction D, the entire moving system 201 moves in the direction D. The movement of the moving system 201 continues until the external force F is consumed as heat by the friction generated between the friction adjusting units and the solid surface.

In the above description, the plate-shaped bodies 205A and 205B serving as brake members are mechanically separated from or pressed against the solid surface 208. However, the present invention is not limited to this. For example, sensors for detecting the rotational directions of the wheels 212A and 212B may be attached. The plate-shaped bodies 205A and 205B may be separated from or pressed against the solid surface 208 by electrically driving the plate-shaped bodies 205A and 105B in the vertical direction on the basis of signals from the sensors.

As described above, in the moving system according to the third embodiment has an effect for converting the oscillation of the spring into rectilinear movement, as in the first and second embodiments. In the first to third embodiments, the force F need not always be mechanically applied but may be magnetically or electrically applied. For example, a force of magnetic flux may be applied to a support made of a magnetic material. Alternatively, a force of electric field may be applied to a charged support.

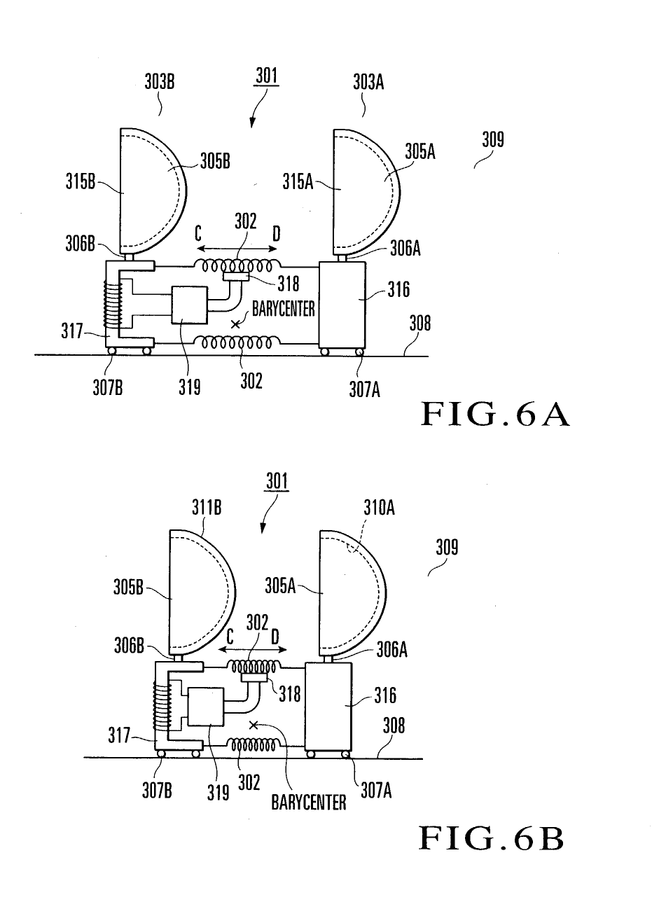

A moving system according to the fourth embodiment of the present invention will be described next with reference to FIGS. 6A and 6B.

FIG. 6A shows a moving system 301 which is set in an equilibrium state without expansion of springs 302 and arranged on a solid surface 308. The moving system 301 according to this embodiment has the pair of springs 302 arranged in parallel and drag adjusting units 303A and 303B fixed to the two ends of each spring 302, as shown in FIG. 6A. The drag adjusting unit 303A has a plunger 316 and a hemispherical body 305A attached to the plunger 316 through a direction control plate 306A. The drag adjusting unit 303B has an electromagnet 317 and a hemispherical body 305B attached to the electromagnet 317 through a direction control plate 306B.

Small casters 307A and 307B are attached to the lower ends of the plunger 316 and electromagnet 317, as in the first embodiment. A strain gauge 318 is attached to one of the springs 302. The output signal from the strain gauge 318 is amplified by an amplifier 319 and supplied to the coil of the electromagnet 317. Parts except the hemispherical bodies and casters of the moving system 301 are shielded from a medium 309 by a shielding member (housing) (not shown). The barycenter of the moving system 301 is located almost at the center of the spring 302.

The hemispherical bodies 305A and 305B have the same shape as that of the hemispherical bodies 5A and 5B of the first embodiment. When the direction control plates 306A and 306B are rotated, the opening directions of openings 315A and 315B can be changed. The operation of the moving system of this embodiment when the openings 315A and 315B of the hemispherical bodies 305A and 305B are directed in a direction C will be described.

When a trigger signal is supplied from a trigger circuit (not shown) to the coil of the electromagnet 317 of the moving system 301 in the equilibrium state, the springs 302 start oscillating in accordance with the natural frequency of the moving system 301. When the springs 302 start oscillating, a signal having the oscillation period of the springs 302 is output from the strain gauge 318 attached to the spring 302 to the amplifier 319. The amplifier 319 amplifies the signal and supplies a current pulse having a predetermined amplitude to the coil of the electromagnet 317. Since the period of the current pulse matches the period of the natural frequency of the moving system 301, self-excited oscillation is induced in the spring 302.

When the spring 302 contracts, the hemispherical body 305A receives, on its inner surface 310A, stress from the medium 309 through the opening 315A, as shown in FIG. 6B. Hence, the drag force from the medium 309 against the movement of the drag adjusting unit 303A in the direction C is large. On the other hand, the hemispherical body 305B only receives, on its outer surface 311B, stress from the medium 309 when the spring 302 contracts. Hence, the drag force from the medium 309 against the movement of the drag adjusting unit 303B in a direction D is small. For this reason, the moving distance of the drag adjusting unit 303A in the direction C is shorter than that of the drag adjusting unit 303B in the direction D. Accordingly, the barycenter of the moving system 301 moves in the direction D.

Subsequently, as in the first embodiment, when the spring 302 repeatedly expands/contracts in accordance with the natural frequency, the moving system moves in the direction D. In the first embodiment, the movement of the moving system stops due to the friction generated between the moving system and the medium. In the fourth embodiment, however, the moving system continuously moves as far as the current pulse is supplied to the coil of the electromagnet 317.

When the direction control plates 306A and 306B are rotated, the moving direction of the moving system 301 is reversed, as in the first embodiment. In the above description, a strain gauge is used to detect the oscillation period of the spring 302. Instead of the strain gauge, any other device such as a piezoelectric element or photodetector capable of detecting the oscillation period or displacement amount can be used.

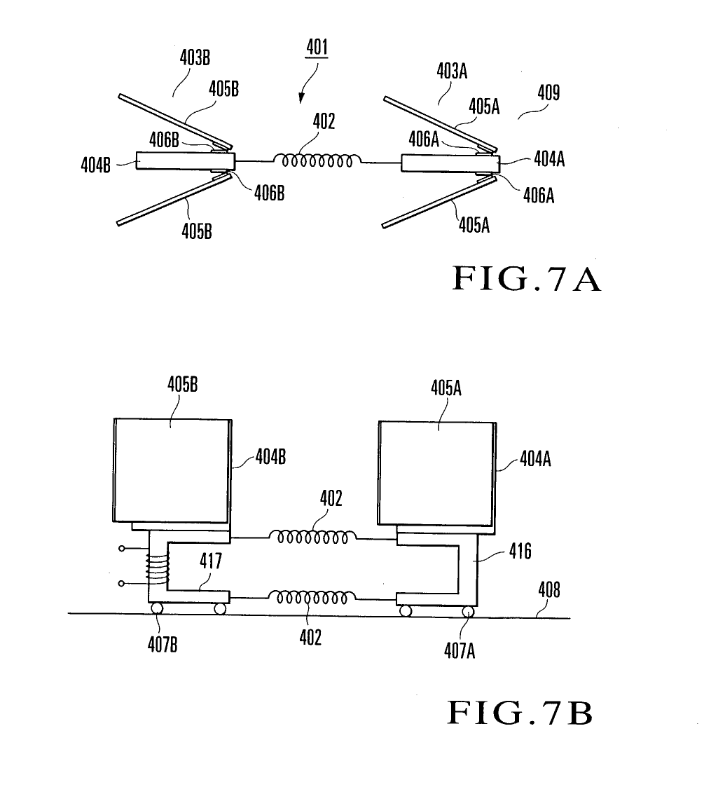

A moving system according to the fifth embodiment of the present invention will be described next with reference to FIGS. 7A and 7B.

A moving system 401 according to this embodiment has springs 402 and drag adjusting units 403A and 403B, as shown in FIG. 7A. The drag adjusting units 403A and 403B have supports 404A and 404B and plate-shaped bodies 405A and 405B attached to the supports 404A and 404B through hinges 406A and 406B. The supports 404A and 404B and plate-shaped bodies 405A and 405B have the same arrangements as in the embodiment shown in FIGS. 2A and 2B, and a description thereof will be omitted.

The support 404A is fixed on a plunger 416 to which small casters 407A are attached, as shown in FIG. 7B. The support 404B is fixed on an electromagnet 417 to which small casters 407B are attached. One end of each spring 402 is connected to the electromagnet 417. The other end of each spring 402 is connected to the plunger 416. When a current flows to the coil of the electromagnet 417, an attracting force is generated between the electromagnet 417 and the plunger 416. Parts except the plate-shaped bodies, supports, and casters of the moving system 401 are shielded from a medium 409 by a shielding member (housing) (not shown).

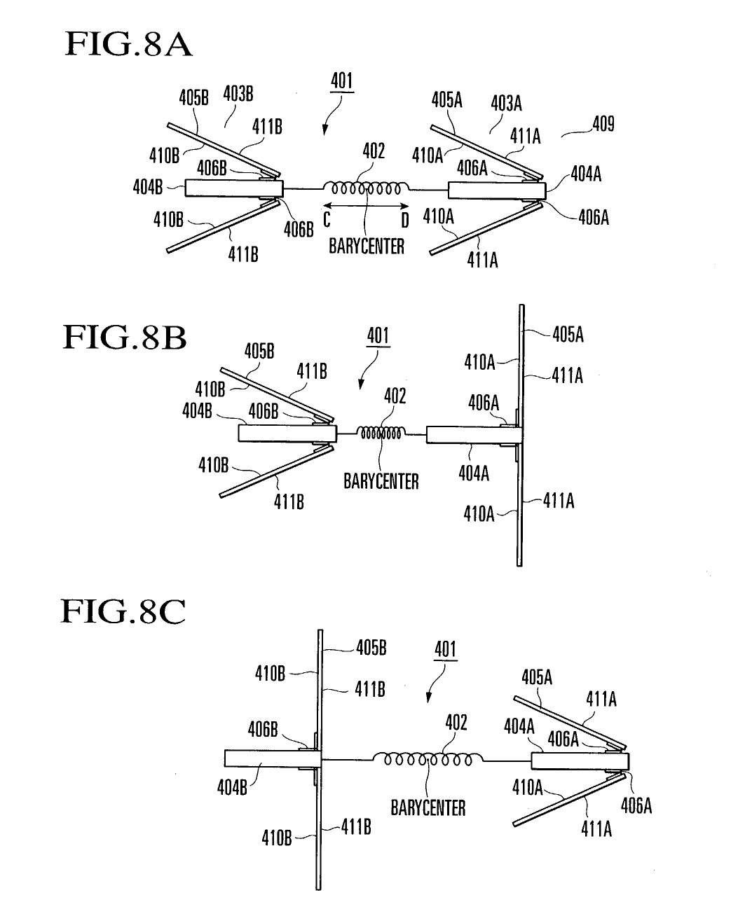

FIG. 8A shows the moving system 401 which is set in an equilibrium state without expansion of the springs 402 and arranged in the medium 409. Referring to FIG. 8A, each of the plate-shaped bodies 405A has inner and outer surfaces 410A and 411A. Each of the plate-shaped bodies 405B has inner and outer surfaces 410B and 411B. The plate-shaped bodies 405A and 405B make an angle close to a minimum angle 2α. The barycenter of the moving system 401 is located almost at the center of the spring 402.

When a predetermined current is supplied to the coil of the electromagnet on which the support 404B of the moving system 401 in the equilibrium state is installed, the springs 402 contract. When the springs 402 contract, the plate-shaped bodies 405A open to the maximum angle of 180° upon receiving, on their inner surfaces 410A, stress from the medium 409, as shown in FIG. 8B. Hence, the movement of the drag adjusting unit 403A in the direction C immediately stops. On the other hand, as the springs 402 contract, the plate-shaped bodies 405B close so the drag received from the medium 409 decreases. More specifically, as the springs 402 contract, the drag received from the medium 409 gradually decreases. Since the contraction of the springs 402 is accelerated, the drag adjusting unit 403B abruptly moves in the direction D. When the drag adjusting unit 403B abruptly moves in the direction D, the springs 402 start expanding due to the repelling force of the springs 402.

When the springs 402 expand, the plate-shaped bodies 405B open to the maximum angle of 180° upon receiving, on their inner surfaces 410B, stress from the medium 409, as shown in FIG. 8C. Hence, the movement of the drag adjusting unit 403B in the direction C immediately stops. On the other hand, as the springs 402 expand, the plate-shaped bodies 405A close so the drag received from the medium 409 gradually decreases. More specifically, as the springs 402 expand, the drag received from the medium 409 decreases. Since the expansion of the springs 402 is accelerated, the drag adjusting unit 403A abruptly moves in the direction D. When the drag adjusting unit 403A abruptly moves in the direction D, the springs 402 start contracting.

Subsequently, as in the fourth embodiment, when the springs 402 repeatedly expand/contract in accordance with the natural frequency, the moving system moves in the direction D. In the fifth embodiment, when an energy is supplied from the magnetic field of the electromagnet 417, the amplitude of the oscillation of the spring 402 exhibits a so-called limit cycle. The fifth embodiment is a modification to the second embodiment in which the oscillation of the spring exhibits a limit cycle. As is apparent, the third embodiment can also be modified such that the oscillation of the spring exhibits a limit cycle.

The spring need not always be oscillated by the magnetic means but may be oscillated by an electrical or/and mechanical means.

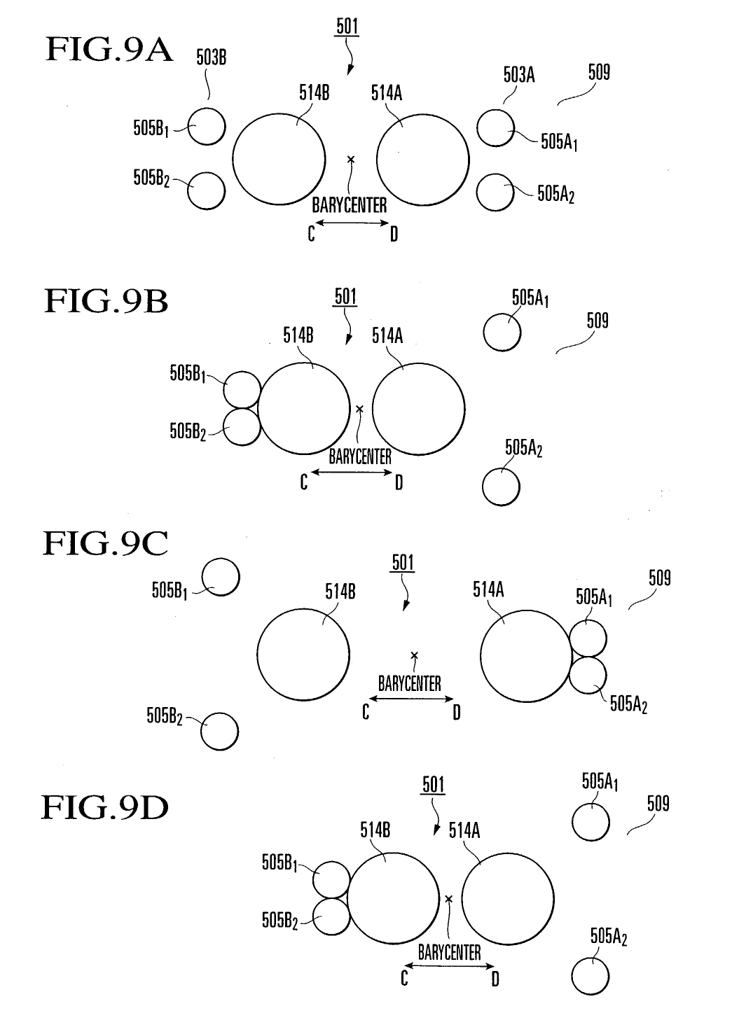

A moving system according to the sixth embodiment of the present invention will be described next with reference to FIGS. 9A to 9D.

A moving system 501 according to this embodiment has a cluster molecule having cores 514A and 514B, side chain portions 505A1 and 505A2 arranged on a D-direction side of the core 514A, and side chain portions 505B1 and 505B2 arranged on a C-direction side of the core 514B, as shown in FIG. 9A. Each of the cores 514A and 514B and side chain portions 505A1, 505A2, 505B1, and 505B2 may be formed from either a single atom or a plurality of atoms. Each of the side chain portions 505A1, 505A2, 505B1, and 505B2 may form one side chain or part of a side chain.

According to the quantum mechanics and solid state theory, oscillation occurs between the cores 514A and 514B. Similarly, oscillation also occurs between the side chain portions 505A1 and 505A2, between the side chain portions 505B1 and 505B2, between the core 514A and the side chain portions 505A1 and 505A2, and between the core 514B and the side chain portions 505B1 and 505B2.

In this embodiment, the cluster molecule has such an oscillation phase that when the space between the cores 514A and 514B contracts, the space between the side chain portions 505A1 and 505A2 and the space between the core 514A and the side chain portions 505A1 and 505A2 expand, and the space between side chain portions 505B1 and 505B2 and the space between the core 514B and the side chain portions 505B1 and 505B2 contract. The cluster molecule also has such an oscillation phase that when the space between the cores 514A and 514B expands, the space between the side chain portions 505A1 and 505A2 and the space between the core 514A and the side chain portions 505A1 and 505A2 contract, and the space between side chain portions 505B1 and 505B2 and the space between the core 514B and the side chain portions 505B1 and 505B2 expand. The side chain portions 505A1 and 505A2 serve as a drag adjusting unit 503A, and the side chain portions 505B1 and 505B2 serve as a drag adjusting unit 503B.

FIG. 9A shows the positions of the cores 514A and 514B and the side chain portions 505A1, 505A2, 505B1, and 505B2 when the oscillation of the cluster molecule is averaged over time.

The moving system according to this embodiment may be formed from a single cluster molecule. Alternatively, the moving system may be constituted by an array structure in which one cluster molecule is defined as a fundamental structure, and a plurality of cluster molecules are arranged in an array in the horizontal direction perpendicular to the C-D direction. Adjacent cluster molecules are bonded to each other by the Van der Waals force.

FIG. 9B shows a state wherein the space between the cores 514A and 514B contracts. As the space between the cores 514A and 514B contracts, the space between the core 514A and the side chain portions 505A1 and 505A2 expands, and the space between the side chain portions 505A1 and 505A2 expands. On the other hand, the space between the core 514B and the side chain portions 505B1 and 505B2 contracts, and the space between the side chain portions 505B1 and 505B2 contracts. The side chain portions 505A1 and 505A2 receive a large drag from a medium 509 because the interval therebetween increases. The side chain portions 505B1 and 505B2 receive a small drag from the medium 509 because the interval therebetween decreases. Hence, the moving distance of the drag adjusting unit 503A to the left side of the drawing surface is shorter than that of the drag adjusting unit 503B to the right side of the drawing surface. For this reason, the barycenter of the moving system 501 moves to the right side of the drawing surface.

Next, as shown in FIG. 9C, the space between the cores 514A and 514B expands. As the space between the cores 514A and 514B expands, the space between the core 514A and the side chain portions 505A1 and 505A2 contracts, and the space between the side chain portions 505A1 and 505A2 contracts. On the other hand, the space between the core 514B and the side chain portions 505B1 and 505B2 expands, and the space between the side chain portions 505B1 and 505B2 expands. The side chain portions 505A1 and 505A2 receive a small drag from the medium 509 because the interval therebetween decreases. The side chain portions 505B1 and 505B2 receive a large drag from the medium 509 because the interval therebetween increases. Hence, the moving distance of the drag adjusting unit 503A to the right side of the drawing surface is longer than that of the drag adjusting unit 503B to the left side of the drawing surface. For this reason, the barycenter of the moving system 501 moves to the right side of the drawing surface.

Next, as shown in FIG. 9D, the space between the cores 514A and 514B contracts. As the space between the cores 514A and 514B contracts, the space between the core 514A and the side chain portions 505A1 and 505A2 expands, and the space between the side chain portions 505A1 and 505A2 expands. On the other hand, the space between the core 514B and the side chain portions 505B1 and 505B2 contracts, and the space between the side chain portions 505B1 and 505B2 contracts. The side chain portions 505A1 and 505A2 receive a large drag from the medium 509 because the interval therebetween increases. The side chain portions 505B1 and 505B2 receive a small drag from the medium 509 because the interval therebetween decreases. Hence, the moving distance of the drag adjusting unit 503A to the left side of the drawing surface is shorter than that of the drag adjusting unit 503B to the right side of the drawing surface. For this reason, the barycenter of the moving system 501 moves to the right side of the drawing surface.

Next, when the space between the cores 514A and 514B expands, the drag adjusting unit 503A largely moves in the direction D while the drag adjusting unit 503B slightly moves in the direction C, as in FIG. 9C. For this reason, the barycenter of the moving system 501 moves in the direction D.

The cluster molecule periodically repeats the above-described contraction/expansion. At this time, since the barycenter of the moving system 501 always moves in the direction D, the entire moving system 501 moves in the direction D. The movement of the moving system 501 continues as far as the cluster molecule continues oscillation.

As described above, the moving system according to the sixth embodiment converts oscillation into rectilinear movement in each molecule.

In the above-described embodiments, drag adjusting units or friction adjusting units are connected to the two ends of a spring or two atoms or molecules. However, the present invention is not limited to this. For example, even when a drag adjusting unit or friction adjusting unit is connected to only one end of a spring, and, e.g., a balancer is connected to the other end, the oscillation of the spring is converted into rectilinear movement, although the moving distance becomes shorter than when drag adjusting units are connected to the two ends.

The present invention has been described above on the basis of the preferred embodiments. The moving system of the present invention is not limited to the above-described embodiments. The present invention also incorporates a moving system for which various changes and modifications are made within the spirit and scope of the invention. For example, the medium in which the moving system moves need not always be a liquid or gas but may be particles or a gel material. The medium is not limited to a specific medium as long as it is a fluid. In addition, the plate-shaped body or hemispherical body that forms a drag adjusting unit may be exchanged with any other body such as a rectangular parallelepiped or a rotating cone as long as it has a shape for receiving a drag force that changes between the contraction mode and expansion mode of the spring. Furthermore, the spring may be exchanged with any other elastic body that oscillates.

As has been described above, according to the present invention, the oscillation of an internal oscillation portion is converted into rectilinear movement through drag adjusting units or friction adjusting units provided at the two ends of the oscillation portion. Hence, the moving system can move in one direction without using any complex power component such as a motor.

In addition, since the drag forces or frictional forces that the two ends of the oscillation portion receive from the medium or solid surface are increased/decreased in reverse directions when the oscillation portion expands/contracts, the moving system can be moved in one direction.

What is claimed is:

1. A moving system comprising:
oscillation means for causing oscillation in accordance with a natural frequency by repeating expansion and contraction; and
conversion means for converting oscillation of said oscillation means into rectilinear movement in one direction.

2. A system according to claim 1, wherein
said conversion means moves in/on one of a medium and a solid surface, and
a drag/frictional force that said conversion means that is moving receives from one of the medium and the solid surface changes between a contraction mode and an expansion mode of said oscillation means.

3. A system according to claim 2, further comprising means for changing the drag/frictional force against said conversion means that is moving.

4. A system according to claim 1, wherein said conversion means is connected to at least one end of said oscillation means in a direction of oscillation.

5. A system according to claim 4, wherein
said oscillation means comprises an expandable spring, and
said conversion means comprises first and second drag units which are connected to two ends of said spring and are movable in a direction of expansion of said spring.

6. A system according to claim 5, wherein
in a contraction mode of said spring, a drag/frictional force against said first drag unit is smaller than that in an expansion mode of said spring, and
in the contraction mode of said spring, the drag/frictional force against said second drag unit is larger than that in the expansion mode of said spring.

7. A system according to claim 5, wherein
in a contraction mode of said spring, a drag/frictional force against said first drag unit is larger than that against said second drag unit, and
in an expansion mode of said spring, the drag/frictional force against said first drag unit is smaller than that against said second drag unit.

8. A system according to claim 1, wherein the oscillation of said oscillation means is started using one of a mechanical energy, an electrical energy, and a magnetic energy.

9. A system according to claim 1, wherein said oscillation means comprises a molecule, and atomic oscillation of the molecule forms the oscillation of said oscillation means.

10. A system according to claim 1, further comprising means for changing a moving direction of said system in a reverse direction.

11. A system according to claim 1, wherein the oscillation of said oscillation means is self-excited oscillation.

12. A system according to claim 1, wherein the oscillation of said oscillation means is a limit cycle.

13. A moving method for a moving system, comprising the steps of:
oscillating an oscillation portion that forms the moving system in accordance with a natural frequency/approximate frequency; and
moving the moving system in one of directions of oscillation of the oscillation portion on the basis of induced oscillation.

14. A method according to claim 13, further comprising the step of, at one and the other ends of the moving system that moves in/on a medium/solid surface, inverting a relationship in magnitude of a drag/frictional force that the moving system receives from the medium/solid surface in contraction and expansion modes of oscillation.
